SEQUENCE LISTING (18 sequences)

SEQ ID NO: 1 - DNA, 219 bp, Synechococcus sp.
atggcagtagagaaagtgaactcctcgtccagcttggccgaagtgatcgatcgcatcttggacaaaggtatcgtggtcgatgcctgggtgcgggtttctttggttgggatcgagctgttggccattgaagcccgcgtcgttgtggcttccgtggaaacctacctgaagtacgctgaggctgtgggtctgacggctactgctgctgctcctgccgtctaa

SEQ ID NO: 2 - protein, 72 aa, Synechococcus sp.
MAVEKVNSSSSLAEVIDRILDKGIVVDAWVRVSLVGIELLAIEARVVVASVETYLKYAEAVGLTATAAAPAV

SEQ ID NO: 3 - DNA, 360 bp, Halobacterium sp.
atggcagatccagcaaacgatcgatctgaacgcgaggaaggcggcgaggacgacgaaacaccgccagcgtccgacgggaacccctcgccgtcggccaattcattcactctctccaacgcgcagacgcgcgcacgagaggcggcacaggacctgttggaacaccagttcgaggggatgatcaaagccgagtcgaacgacgaaggctggcggaccgtcgtcgaagtcgtcgaacggaacgccgtacccgatacacaagacatcatcggtcgctacgagatcacgcttgacgggacgggggacgtcaccggctacgagctcctagaacgctatcgtcggggcgacatgaaagaggaactgtag

SEQ ID NO: 4 - protein, 119 aa, Halobacterium sp
MADPANDRSEREEGGEDDETPPASDGNPSPSANSFTLSNAQTRAREAAQDLLEHQFEGMIKAESNDEGWRTVVEVVERNAVPDTQDIIGRYEITLDGTGDVTGYELLERYRRGDMKEEL

SEQ ID NO: 5 - DNA, 768 bp, Bacillus megaterium
atgagtgaaacaaacgaaacaggtatttatatttttagcgccattcaaacggataaagacgaagaatttggcgccgtggaagtagaaggaacaaaagctgaaacatttttgattcgctacaaagacgcggctatggtagcagctgaagtaccgatgaaaatttatcatcctaatcgccaaaatttattaatgcatcaaaacgcagtagcagcgattatggacaagaacgatacggttattccaatcagctttgggaatgtattcaaatcaaaagaagacgtaaaagttcttttggaaaacctttatccgcagtttgaaaagctgtttccagcgatcaaaggaaaaattgaagtcggtttaaaagtaattgggaaaaaagaatggcttgagaaaaaagtaaacgaaaatcctgaacttgagaaagtatcagcatccgtaaaaggaaaatcagaagcagccggttattatgagcgtattcaacttggaggaatggctcaaaagatgtttacttccctgcaaaaagaagtcaagacagatgtattttctccgcttgaagaagcagcggaagcagcaaaagcaaatgagccaacgggcgaaacgatgcttttaaacgcgtctttcttaattaaccgagaagatgaagcgaagtttgatgaaaaagtaaatgaagcgcatgaaaactggaaagacaaagccgattttcattacagcggtccttggcctgcttataattttgtgaacattcgcctaaaagtagaagagaaataa

SEQ ID NO: 6 - protein, 255 aa, Bacillus megaterium
MSETNETGIYIFSAIQTDKDEEFGAVEVEGTKAETFLIRYKDAAMVAAEVPMKIYHPNRQNLLMHQNAVAAIMDKNDTVIPISFGNVFKSKEDVKVLLENLYPQFEKLFPAIKGKIEVGLKVIGKKEWLEKKVNENPELEKVSASVKGKSEAAGYYERIQLGGMAQKMFTSLQKEVKTDVFSPLEEAAEAAKANEPTGETMLLNASFLINREDEAKFDEKVNEAHENWKDKADFHYSGPWPAYNFVNIRLKVEEK

SEQ ID NO: 7 - DNA, 237 bp, Synechococcus sp.
atggtttggcaattgttgacttggccggcccaaagtttgctttggctagcagagcagatccaagaacgcgccgaagcacagctggatagcaaagaaaacctgcaaaaagaacttacggccctgcaaattcagctagatttgggagaaattgacgaagaaacctacgcccgccgagaagaggagattttattggctctggaagccttaacccaagcagaaggagaagccgaagcatag

SEQ ID NO: 8 - protein, 78 aa, Synechococcus sp.
MVWQLLTWPAQSLLWLAEQIQERAEAQLDSKENLQKELTALQIQLDLGEIDEETYARREEEILLALEALTQAEGEAEA

SEQ ID NO: 9 - DNA, 336 bp, Synechococcus sp.
gtgccgattagctctcaacccttgaccacggctactcacggctcctcgctggccgatgtgttggagcgggtgctggacaagggcattgtgatcgccggagacatcaccgtttcggtgggcaatgtggagttgctgaatgtgcgcattcgcctgctgatttcttcggtggataaggccaaggagatcggcatcaattggtgggagtcggatccctatctcaacagccaggcgcgggagctgctggaagccaaccgacagctcatgcagcgcgttgccgaattggaaagacagcttgcccaagctctgccccaggggggaaagggaacggacccatag

SEQ ID NO: 10 - protein, 111 aa, Synechococcus sp.
MPISSQPLTTATHGSSLADVLERVLDKGIVIAGDITVSVGNVELLNVRIRLLISSVDKAKEIGINWWESDPYLNSQARELLEANRQLMQRVAELERQLAQALPQGGKGTDP

SEQ ID NO: 11 - DNA, 1299 bp, Synechococcus sp.
atggagttcgctaggccccggcgaatgtccccccgcattctggttctggattttgatggtgtgctctgcgatgggcgggcggagtattttgcctcttcctgccgcgtttgtgctcaggtgtggggcttggctcctgctcagctagagccgctgcgtcctgcttttgaccgtctgcgcccgctgattgagaccggctgggagatgcctctgttgttgtgggggctacaggaagggatccgggaggaagacttgcgccaagactggcccagctggcggcagcggttgttgcagcagtcagggatccctgccctctctctaatccaagcgttggatcgggtgcgggatcgctggattgcagaggatctgcaggggtggctggggctgcaccggttttatccgggggtggcggcctggatgcgccagcttcaggctgccggggagccgcgcttggccatcctcagcaccaaagagggacggttcatccagcagctcttgggccgagcagggatccaactgccgcgccaccgcattctgggcaaggaagtgcgcgcccccaaggccaccactttacagcggctactggctgccgcccaactgccggctgaggagctgtggtttgtggaggatcgcctgcaaacgctgcgccaggtgcagagggtgccggagctggagcaggttctcttgtttttggccgactggggctacaacctaccagaggaaagggaagaggccgctcgggatccccgtctccatttgctcagcctggaacagctttgtcagccctttgaccgttggattgcttctcctcccccgccgcgcttttctatcagtcccgccagctgggaagacttgagccagactcggcccacccctggccggaaacgcccggaagctggtttggcctctctggtgctgaccttggtggagctgttgcggcagttgatggaggcgcaggtggtgcggcaaatggaggctgagcgcctttctgcagagcagattgagcgggccggcagcagcctacaagccttgcgggagcaaattcgacaaatctgcagcctgttggagatcgacccagcggatttgaacctggagctcggagatctgggcaccctcctgccccgccagggggactactaccccggacaaccccaccgcgagggatccgtgctggaactgttggatcggctgatccacaccggcatcgtcatcgatggggagatcgacctggggctggcggacttggatctgatccacgcccgcctgaagttggtgcttacctccagcgccaagctctactga

SEQ ID NO: 12 - protein, 432 aa, Synechococcus sp.
MEFARPRRMSPRILVLDFDGVLCDGRAEYFASSCRVCAQVWGLAPAQLEPLRPAFDRLRPLIETGWEMPLLLWGLQEGIREEDLRQDWPSWRQRLLQQSGIPALSLIQALDRVRDRWIAEDLQGWLGLHRFYPGVAAWMRQLQAAGEPRLAILSTKEGRFIQQLLGRAGIQLPRHRILGKEVRAPKATTLQRLLAAAQLPAEELWFVEDRLQTLRQVQRVPELEQVLLFLADWGYNLPEEREEAARDPRLHLLSLEQLCQPFDRWIASPPPPRFSISPASWEDLSQTRPTPGRKRPEAGLASLVLTLVELLRQLMEAQVVRQMEAERLSAEQIERAGSSLQALREQIRQICSLLEIDPADLNLELGDLGTLLPRQGDYYPGQPHREGSVLELLDRLIHTGIVIDGEIDLGLADLDLIHARLKLVLTSSAKLY

SEQ ID NO: 13 - DNA, 846 bp, Halobacterium sp
atgactgaccaccggcccagcccggaagaagagcagaccacagcgaacgaggaacggacggtcagcaacggccgctatctatactgcgtggtcgataccacgtcgtcggaatcggcgaccctgtccacgaccggggtcgacgacaaccctgtctacgtcgtcgaggccgatggcgtgggcgccgtcgtccatgactgtgagacggtctacgagacggaagacctcgaacaggtgaagcgatggctggtcacgcaccagcaggtcgtcgacgcggcgagcgacgcgttcggtacgccgctgccgatgcgattcgacacggtcctcgagggcggtgatgcgagtatcgaacggtggttagaagaccactacgagggcttccgcgacgaattagcgtcgttcgcgggagtgtgggagtatcgaatcaatctgttgtgggattccgcaccgttcgaggagaccatcgcagaccgagacgaccggctccgagaactacgacagcgccagcaacaatcgggcgcagggaaaaagttcctcctcgagaaacagtccgatcagcgactccaagagctgaaacgagagcgccggacggaactagcagatcaactgaaagaggccattaccccggtcgtgaacgacctgaccgaacaggacacgaatacgccgctacaggacgaacactcgtccatcgagaaagaacagatcgtgcggttcgccgttctcgcggacgaggacgacgagaccgctctcggtgatcgattggatacgatcgtcgaacacgagggtgtagagatcagattcacggggccgtggccaccgtacacgttcgcgccagatattggtaaataa

SEQ ID NO: 14 - protein, 281 aa, Halobacterium sp.
MTDHRPSPEEEQTTANEERTVSNGRYLYCVVDTTSSESATLSTTGVDDNPVYVVEADGVGAVVHDCETVYETEDLEQVKRWLVTHQQVVDAASDAFGTPLPMRFDTVLEGGDASIERWLEDHYEGFRDELASFAGVWEYRINLLWDSAPFEETIADRDDRLRELRQRQQQSGAGKKFLLEKQSDQRLQELKRERRTELADQLKEAITPVVNDLTEQDTNTPLQDEHSSIEKEQIVRFAVLADEDDETALGDRLDTIVEHEGVEIRFTGPWPPYTFAPDIGK

SEQ ID NO: 15 - DNA, 255 bp, Halobacterium sp.
atggagccaacaaaagacgagacacacgcgatcgttgagttcgtcgacgtgttactgcgcgacggagccgtgattcaagcggacgtgatcgtgacggtcgccgacattcccctgatcgggatcagcctccgggcagcgattgctggcatgaccaccatgacggagtacggcctgttcgaggagtgggatgctgcgcatcgacaacagagcgaagcgttcacgacctcgcccactgccgatcggcgagaggactga

SEQ ID NO: 16 - protein, 84 aa, Halobacterium sp
MEPTKDETHAIVEFVDVLLRDGAVIQADVIVTVADIPLIGISLRAAIAGMTTMTEYGLFEEWDAAHRQQSEAFTTSPTADRRED

SEQ ID NO: 17 - DNA, 820 bp, Chlamydomonas sp.
ggatcccacacacctgcccgtctgcctgacaggaagtgaacgcatgtcgagggaggcctcaccaatcgtcacacgagccctcgtcagaaacacgtctccgccacgctctccctctcacggccgaccccgcagcccttttgccctttcctaggccaccgacaggacccaggcgctctcagcatgcctcaacaacccgtactcgtgccagcggtgcccttgtgctggtgatcgcttggaagcgcatgcgaagacgaaggggcggagcaggcggcctggctgttcgaagggctcgccgccagttcgggtgcctttctccacgcgcgcctccacacctaccgatgcgtgaaggcaggcaaatgctcatgtttgcccgaactcggagtccttaaaaagccgcttcttgtcgtcgttccgagacatgttagcagatcgcagtgccacctttcctgacgcgctcggccccatattcggacgcaattgtcatttgtagcacaattggagcaaatctggcgaggcagtaggcttttaagttgcaaggcgagagagcaaagtgggacgcggcgtgattattggtatttacgcgacggcccggcgcgttagcggcccttcccccaggccagggacgattatgtatcaatattgttgcgttcgggcactcgtgcgagggctcctgcgggctggggagggggatctgggaattggaggtacgaccgagatggcttgctcggggggaggtttcctcgccgagcaagccagggttaggtgttgcgctcttgactcgttgtgcattctaggaccccactgctactcacaacaagcc

SEQ ID NO: 18 - DNA, 5298 bp, Artificial sequence
  Synthetic sequence
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggcttgacatgattggtgcgtatgtttgtatgaagctacaggactgatttggcgggctatgagggcgcgggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggatggccactccgccctccccggtgctgaagaatttcgaagcatggacgatgcgttgcgtgcactgcggggtcggtatcccggttgtgagtgggttgttgtggaggatggggcctcgggggctggtgtttatcggcttcggggtggtgggcgggagttgtttgtcaaggtggcagctctgggggccggggtgggcttgttgggtgaggctgagcggctggtgtggttggcggaggtggggattcccgtacctcgtgttgtggagggtggtggggacgagagggtcgcctggttggtcaccgaagcggttccggggcgtccggccagtgcgcggtggccgcgggagcagcggctggacgtggcggtggcgctcgcggggctcgctcgttcgctgcacgcgctggactgggagcggtgtccgttcgatcgcagtctcgcggtgacggtgccgcaggcggcccgtgctgtcgctgaagggagcgtcgacttggaggatctggacgaggagcggaaggggtggtcgggggagcggcttctcgccgagctggagcggactcggcctgcggacgaggatctggcggtttgccacggtcacctgtgcccggacaacgtgctgctcgaccctcgtacctgcgaggtgaccgggctgatcgacgtggggcgggtcggccgtgcggaccggcactccgatctcgcgctggtgctgcgcgagctggcccacgaggaggacccgtggttcgggccggagtgttccgcggcgttcctgcgggagtacgggcgcgggtgggatggggcggtatcggaggaaaagctggcgttttaccggctgttggacgagttcttctgagggacctgatggtgttggtggctgggtagggttgcgtcgcgtgggtgacagcacagtgtggacgttgggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcgggtacccagcttttgttccctttagtgagggttaattgcgcgcttggcgtaatcatggtcatagctgtttcctgtgtgaaattgttatccgctcacaattccacacaacatacgagccggaagtctagacggcggggagctcgctgaggcttgacatgattggtgcgtatgtttgtatgaagctacaggactgatttggcgggctatgagggcgcgggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggatggccactccgccctccccggtgctgaagaatttcgaaattaaccctcactaaagggaacaaaagctgggtaccgggccccccctcgaggtcgacggtatcgataagcttgatatcgaattcctgcagcccgggggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcgggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

(19) United States
(12) Patent Application Publication
(10) Pub. No.: US 2014/0234904 A1
(43) Pub. Date: Aug. 21, 2014

(54) METHOD FOR HARVESTING PHOTOSYNTHETIC UNICELLS USING GENETICALLY INDUCED FLOTATION

(71) Applicant: University of Wyoming, Laramie, WY (US)

(72) Inventors: Stephen K. Herbert, Laramie, WY (US); Levi G. Lowder, Laramie, WY (US)

(73) Assignee: University of Wyoming, Laramie, WY (US)

(21) Appl. No.: 14/349,039

(22) PCT Filed: Oct. 5, 2012
(86) PCT No.: PCT/US2012/058884

§ 371 Date: Apr. 1, 2014

Related U.S. Application Data
(60) Provisional application No. 61/544,204, filed on Oct. 6, 2011.

(51) Int. Cl. C12N 15/74; C12P 21/00

(52) U.S. Cl. CPC C12N 15/74; C12P 21/00
USPC 435/69.1; 435/320.1; 435/252.3

(57) ABSTRACT

Methods for the harvesting of photosynthetic unicellular organisms are provided, including the formation and expression or overexpression of gas vesicles or vacuole proteins in photosynthetic unicellular organisms. DNA constructs as well as methods for integration of the DNA constructs into the genomes of photosynthetic unicellular organisms for the formation and expression or overexpression of gas vesicles or vacuole expression proteins in unicellular organisms are also disclosed.

### CROSS REFERENCE TO RELATED APPLICATIONS

This application claims priority to and the benefit under 35 U.S.C. 3.71 of PCT/US2012/058884, filed on Oct. 5, 2012 and U.S. Provisional Application No. 61/544,204 filed Oct. 6, 2011, the entire contents of which are incorporated herein by reference for all purposes.

### SUBMISSION OF SEQUENCE LISTING

The Sequence Listing associated with this application is filed in electronic format via EFS-Web and is hereby incorporated by reference into the specification in its entirety.

### BACKGROUND

All publications cited in this application are herein incorporated by reference.

Algal biomass production has a huge potential as a feedstock for human and animal food, as well as for use in liquid fuels, plastics, soil amendments, and many other useful materials. Among many benefits, the ability to produce algae cheaply at large scales allows the creation of agricultural industries in areas with limited amounts of arable land and other limited resources. Algal biomass also has the added benefit of lowering the cost of sequestration of CO₂, NOx, and SO₂ from the burning of fossil fuels, and the generation of renewable biofuels with little impact on traditional food production. Traditional techniques for harvesting algal biomass include centrifugation, filtration, and chemical flocculation.

Various phyla of bacteria, including many cyanobacteria, are capable of assembling gas vesicles for controlling buoyancy in aquatic habitats. These vesicles are assembled from protein monomers that self-assemble into conical filaments. The proteinaceous filaments are capable of blocking the diffusion of water molecules into the vesicle lumen but allow the diffusion of gasses into the filament space, creating a gas-filled compartment that increases the positive buoyancy of cells to allow for harvesting without the need for centrifugation, filtration, and chemical flocculation.

The foregoing examples of related art and limitations related therewith are intended to be illustrative and not exclusive, and they do not imply any limitations on the inventions described herein. Other limitations of the related art will become apparent to those skilled in the art upon a reading of the specification and a study of the drawings.

### SUMMARY

It is to be understood that the present invention includes a variety of different versions or embodiments, and this Summary is not meant to be limiting or all-inclusive. This Summary provides some general descriptions of some of the embodiments, but may also include some more specific descriptions of other embodiments.

An embodiment of the present invention may comprise DNA constructs for the expression of proteins in a photosynthetic unicellular organism, where the expressed protein is for the formation and expression or overexpression of gas vesicles protein. Such DNA constructs may be represented as Pro1-gvpAO-SM1-Pro2-gvpFGJKLM-SM2, Pro-HetGVP-SM, psbD-HetGVP-psbD wherein Pro, Pro1, Pro2 and psbD are an inducible and/or constitutive promoter and regulatory regions used for homologous recombination into plastid genomic loc, gvpAO, gvpFGJKLM and HetGVP are gas vesicle formation and expression or overexpression genes, and SM, SM1 and SM2 are selectable markers such as a fluorescent protein sequence.

An embodiment may further comprise a transgenic photosynthetic unicellular organism having a DNA construct stably integrated into the organism's nuclear genome or the organism's chloroplast genome under conditions suitable for an expression of the DNA construct in the organism, wherein the expressed protein is a gas vesicle formation and expression or overexpression protein.

An embodiment of the present invention may further comprise a method for producing a transgenic photosynthetic unicellular organism expressing or overexpressing a gas vesicle expression protein which comprises growing a transgenic photosynthetic unicellular organism having a DNA construct stably integrated into the organism's nuclear genome or chloroplast genome under conditions suitable for the formation and expression of the DNA construct in the transgenic photosynthetic unicellular organism, and wherein the expressed or overexpressed protein is a gas vesicle expression protein.

In addition to the examples, aspects and embodiments described above, further aspects and embodiments will become apparent by reference to the drawings and by study of the following descriptions, any one or all of which are within the invention. The summary above is a list of example implementations, not a limiting statement of the scope of the invention.

### BRIEF DESCRIPTION OF THE FIGURES

The accompanying drawings, which are incorporated herein and form a part of the specification, illustrate some, but not the only or exclusive, example embodiments and/or features. It is intended that the embodiments and figures disclosed herein are to be considered illustrative rather than limiting.

FIG. 1 is a map of a DNA construct, represented as Pro1-gvpAO-SM1-Pro2-gvpFGJKLM-SM2 that includes (from 5′ to 3′), a first promoter; the gas vesicle proteins gvpA and gvpO, a first selectable marker, a second promoter, a second group of gas vesicle proteins comprising gvpF, gvpO, gvpJ, gvpK, gvpL, gvpM and a second selectable marker.

FIG. 2 is a map of a DNA construct, represented as Pro-HetGVP-SM that includes (from 5′ to 3′), promoter; a heterologous operon comprising a series of gas vesicle formation proteins gvpA, gvpO, gvpF, gvpO, gvpJ, gvpK, gvpL and gvpM and a selectable marker.

FIG. 3 is a map of a DNA construct, represented as psbD-HetGVP-psbD that includes (from 5′ to 3′) the 5′ end of the psbD chloroplast gene with native promoters, a heterologous operon coding the gas vesicle formation genes gvpA, gvpO, gvpF, gvpO, gvpJ, gvpK, gvpL and gvpM and the 3′ end of the psbD chloroplast gene.

### BRIEF DESCRIPTION OF THE SEQUENCE LISTINGS

SEQ ID NO: 1 discloses the nucleic acid sequence for the gvpA gas vesicle synthesis protein GvpA [Synechococcus sp. JA-2-3B′a(2-13)] Gene ID: 3901105 sequence (GENBANK Accession No. NC_{—}007776).

SEQ ID NO: 2 discloses the protein sequence for the gvpA gas vesicle synthesis protein GvpA [Synechococcus sp. JA-2-3B′a(2-13)] (GENBANK Accession number YP_{—}478051).

SEQ ID NO: 3 discloses the nucleic acid sequence of the gvpO gas vesicle protein GvpO [Halobacterium sp. NRC-1] Gene ID: 1446788 sequence (GENBANK Accession NC_{—}001869).

SEQ ID NO: 4 discloses the protein sequence of gvpO gas vesicle protein GvpO [Halobacterium sp. NRC-1] Gene ID: 1446788 sequence (GENBANK Accession NP_{—}045973.1).

SEQ ID NO: 5 discloses the nucleic acid sequence of the gvpF gas vesicle protein GvpF [Bacillus megaterium QM B1551] Gene ID: 8987735 sequence (GENBANK Accession NC_{—}014019).

SEQ ID NO: 6 discloses the protein sequence of the gvpF gas vesicle protein GvpF [Bacillus megaterium QM B1551] Gene ID: 8987735 sequence (GENBANK Accession YP_{—}003563753).

SEQ ID NO: 7 discloses the nucleic acid sequence of gvpG gas vesicle protein G [Synechococcus sp. JA-2-3B′a(2-13)] Gene ID: 3902627 sequence (GENBANK Accession NC_{—}007776).

SEQ ID NO: 8 discloses the protein sequence of the gvpG gas vesicle protein G [Synechococcus sp. JA-2-3B′a(2-13)] Gene ID: 3902627 sequence (GENBANK Accession YP_{—}478345).

SEQ ID NO: 9 discloses the nucleic acid sequence for gvpJ gas vesicle protein J [Synechococcus sp. JA-2-3B′a(2-13)] Gene ID: 3901101 sequence (GENBANK Accession NC_{—}007776).

SEQ ID NO: 10 discloses the protein sequence of the gvpJ gas vesicle protein J [Synechococcus sp. JA-2-3B′a(2-13)] Gene ID: 3901101 sequence (GENBANK Accession YP_{—}478047).

SEQ ID NO: 11 discloses the nucleic acid sequence for the gvpK HAD hydrolase-like protein/gas vesicle protein K [Synechococcus sp. JA-2-3B′a(2-13)] Gene ID: 3901471 sequence (GENBANK Accession No. NC_{—}007776).

SEQ ID NO: 12 discloses the protein sequence for the gvpK HAD hydrolase-like protein/gas vesicle protein K [Synechococcus sp. JA-2-3B′a(2-13)] Gene ID: 3901471 sequence (GENBANK Accession No. YP_{—}477701.1).

SEQ ID NO: 13 discloses the nucleic acid sequence for gvpL gas vesicle protein GvpL [Halobacterium sp. NRC-1] Gene ID: 1446776 sequence (GENBANK Accession No. NC_{—}001869).

SEQ ID NO: 14 discloses the protein sequence for the gvpL gas vesicle protein GvpL [Halobacterium sp. NRC-1] Gene ID: 1446776 sequence (GENBANK Accession No. NP_{—}045961).

SEQ ID NO: 15 discloses the nucleic acid sequence for the gvpM gas vesicle protein GvpM [Halobacterium sp. NRC-1] Gene ID: 1446775 sequence (NCBI Reference Sequence NC_{—}001869).

SEQ ID NO: 16 discloses the protein sequence for the gvpM gas vesicle protein GvpM [Halobacterium sp. NRC-1] Gene ID: 1446775 sequence (NCBI Reference Sequence NP_{—}045960.1).

SEQ ID NO: 17 discloses the nucleic acid sequence for the PSAD promoter.

SEQ ID NO: 18 discloses the nucleic acid sequence for the RbcS2 promoter flanked by enhancer elements of Hsp70A and RbcS2 intron 1 (“Hsp70A/RbcS2”).

### DETAILED DESCRIPTION

Embodiments of the present invention include DNA constructs as well as methods for integration of the DNA constructs into photosynthetic eukaryotic and prokaryotic unicells, including but not limited to cyanobacteria, for the transgenic and cisgenic formation and expression of gas vesicle or vacuole genes for the heterologous formation and expression or overexpression of gas vesicle or vacuole proteins in photosynthetic unicellular organisms. A “construct” is an artificially constructed segment of DNA that may be introduced into a target unicellular organism.

Embodiments also include methods for harvesting photosynthetic unicells at large scales for low cost biomass production including genetically modify cyanobacteria to overexpress native genes for gas vacuoles or gas vesicles. The genetic modification upon genetic induction such that buoyancy is increased and flotation is accomplished for easy separation of cells from the growth medium. A second method includes genetically modify cyanobacteria to overexpress heterologous genes for gas vacuoles or vesicles in the same manner as the former strategy. A third method includes genetically modifying eukaryotic unicellular algae for inducible expression of heterologous genes for gas vacuoles or vesicles such that buoyancy is increased and flotation is accomplished for easy separation from growth medium.

As used herein, the term “expression” includes the process by which information from a gene is used in the synthesis of a functional gene product, such as the formation and expression of gas vesicle or vacuole proteins in eukaryotic and prokaryotic unicellular organisms. These products are often proteins, but in non-protein coding genes such as rRNA genes or tRNA genes, the product is a functional RNA. The process of gene expression is used by all known life, i.e., eukaryotes (including multicellular organisms), prokaryotes (bacteria and archaea), and viruses, to generate the macromolecular machinery for life. Several steps in the gene expression process may be modulated, including the transcription, up-regulation, RNA splicing, translation, and post translational modification of a protein.

As used herein, the term “operon” is a group of closely linked genes responsible for the synthesis of one or a group of enzymes which are functionally related as members of one enzyme system.

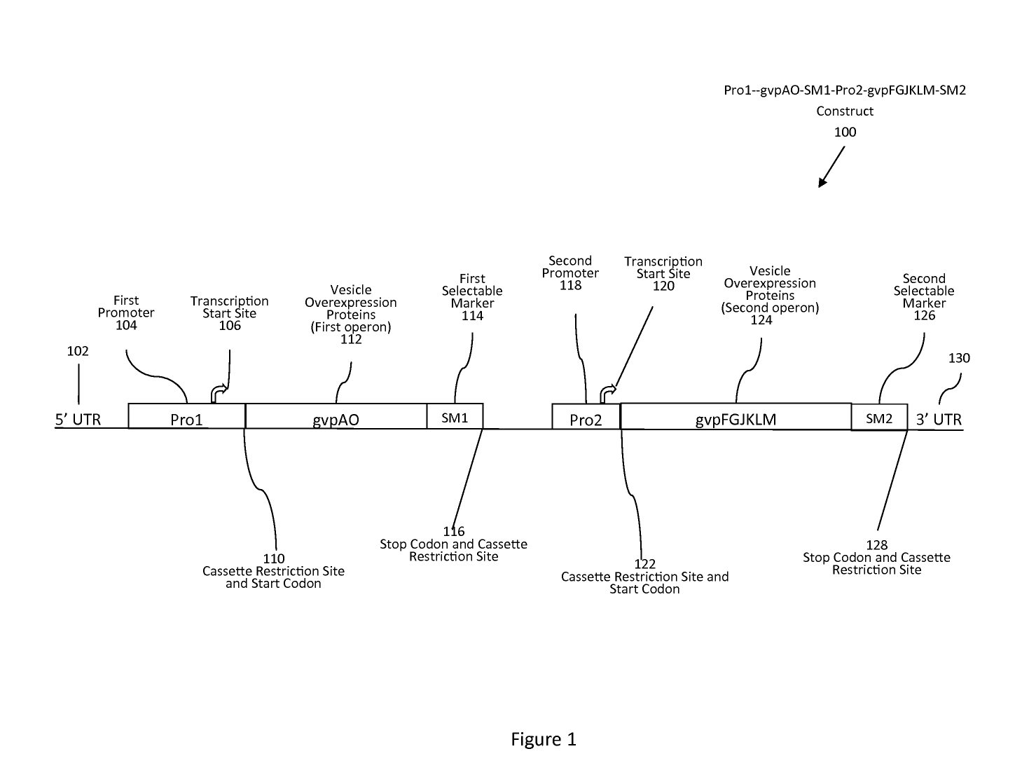

As shown in FIG. 1, a construct comprising two operons to ensure the induced overexpression of gas vesicles in buoyant prokaryotic unicellular organisms, including but not limited to cyanobacteria is generally represented as Pro1-gvpAO-SM 1-Pro2-gvpFGJKLM-SM2 100, where starting at the 5′ UTR 102 an inducible transcriptional promoter such as IPTG inducible Ptrc promoter and a pEL5 translational enhancing sequence is provided as Pro1 104 with a transcription start site 106. The first operon gvpAO 112 comprises the gas vesicle formation and expression or overexpression proteins GvpA (SEQ ID NO. 1) and GvpO (SEQ. ID NO: 3) where the operon has a restriction site and start codon 110 on the 5′ end of the gas vesicle operon and each protein coding sequence of said operon has a ribosomal binding site preceding the open reading frame (ORF) such that individual coding sequences of the operon can be translated independently of the operon. SM1, 114 is a first selectable marker such as a bleomycin (Ble) resistance marker, a hygromycin resistance marker, hygromycin, the paromomycin resistance marker or a fluorescent fusion protein yellow fluorescent protein (YFP), a cyan fluorescent protein (CFP), a red fluorescent protein (mRFP).). A stop codon and 3′ cassette restriction site 116 provides the translational termination on the first operon and after each protein coding ORF within said operon. The construct also contains a second inducible transcriptional promoter such as IPTG inducible Ptrc promoter and a pEL5 translational enhancing sequence is provided as Pro2 118 with a transcription start site 120. The second operon, gvpFGJKLM 124 are the gas vesicle proteins GvpF (SEQ ID NO: 5), GvpG (SEQ ID NO: 7), GvpJ (SEQ ID NO: 9), the HAD hydrolase-like protein/gas vesicle protein GvpK (SEQ ID NO: 11), GvpL (SEQ ID NO:13) and the gas vesicle protein GvpM (SEQ ID NO: 15) where the second operon has a restriction site and start codon 122 on the 5′ end of the second set of gas vesicle proteins 122. SM2, 126 is a second and different selectable marker from the first selectable marker SM1 114 such as a bleomycin (Ble) resistance marker, a hygromycin resistance marker, hygromycin, the paromomycin resistance marker (aph VIIIsr) or a fluorescent fusion protein yellow fluorescent protein (YFP), a cyan fluorescent protein (CFP), a red fluorescent protein (mRFP). A stop codon and 3′ cassette restriction site 128 provides the transcription termination on the 3′UTR 130. Each of these components is operably linked to the next, i.e., the first promoter is operably linked to the 5′ end of the first operon comprising the gvpAO gas vesicle protein coding sequences encoding the gvpAO gas vesicle proteins. The first operon gvpAO gas vesicle coding sequences are operably linked to the first selectable marker coding sequence. The first selectable marker coding sequence is operably linked to the second promoter coding sequence. The second promoter coding sequence is operably linked to the 5′ end of the second operon gvpFGJKLM gas vesicle expression protein sequences encoding the gvpFGJKLM gas vesicle expression proteins and the second operon gvpFGJKLM gas vesicle expression protein coding sequences are operably linked to the 5′ end of the second selectable marker coding sequence. The DNA construct Pro1-gvpAO-SM1-Pro2-gvpFGJKLM SM2 100 is then integrated into an expression vector, such as the expression vector pSK.KmR or pEL5 or expressed from a separate plasmid or plasmids and organisms overexpressing a gas vesicle protein are then generated including but not limited to Synechococcus, Aphanizomenon, Anadaena, Gleotrichia, Oscillatoria, Halobacterium, Calothrix and Nostoc. The DNA construct Pro1-gvpAO-SM1-Pro2-gvpFGJKLM SM2 100 for the transgenic and cisgenic expression of the gvpAOFGJKLM genes using expression vectors based on pSI105, pSK.KmR and pEL5 using the IPTG inducible Ptrc promoter and pEL5 translational enhancing may also be used for heterologous gas vesicle expression in model and commonly used cyanobacteria that are not yet known to produce gas vesicles or vacuoles, including but not limited to Arthrospira spp. or Spirulina spp., Synechococcus elongatus 7942, Synechococcus spp., Synechosystis spp. PCC 6803, Synechosystis spp., and Spirulina plantensis sequences (see Lan, E I and Liao, J C, Metabolic Engineering 13:353-363 (2011)).

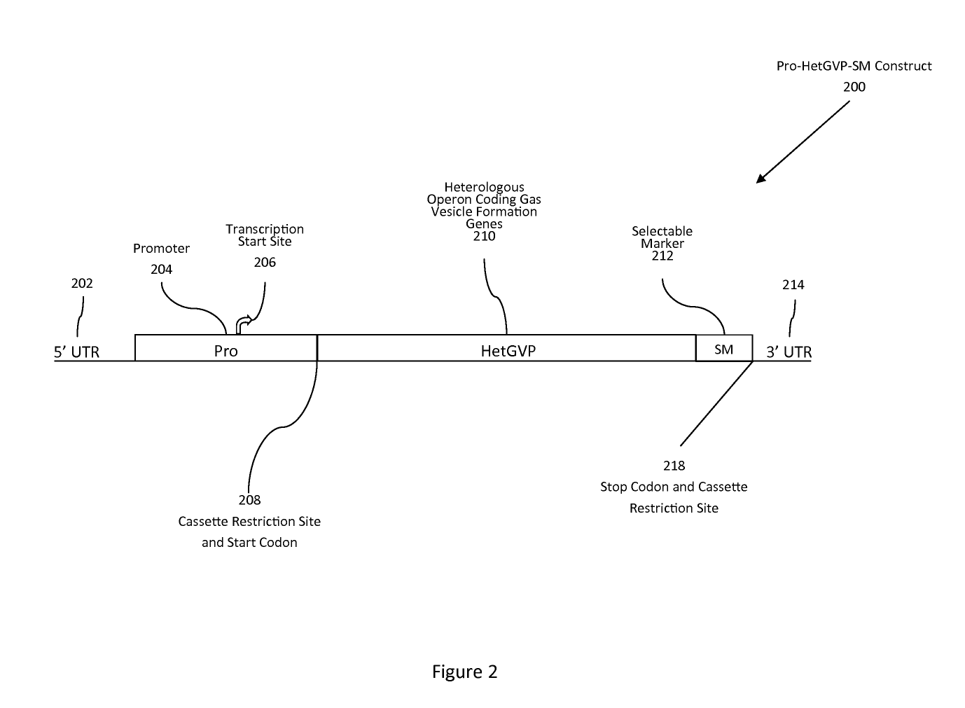

As shown in FIG. 2, a construct comprising a single operon for the induced heterologous formation and expression of gas vesicles in a photosynthetic eukaryotic unicellular algae is generally represented as Pro-HetGVP-SM 200, where starting at the 5′ UTR 202 promoters such as the RbcS2 promoter (SEQ ID NO: 18) or a promoter with an associated regulatory element promoter such as the PSAD promoter (SEQ ID NO: 17) is provided as Promoter (Pro) 204 with the transcription start site 206. HetGVP 210 is a single operon comprising the gas vesicle synthesis protein GvpA (SEQ ID NO. 1), the gas vesicle protein GvpO (SEQ. ID NO: 3), the gas vesicle protein GvpF (SEQ ID NO: 5), the gas vesicle protein GvpG (SEQ ID NO: 7), the gas vesicle protein GvpJ (SEQ ID NO: 9), the HAD hydrolase-like protein/gas vesicle protein GvpK (SEQ ID NO: 11), the gvpL gas vesicle protein GvpL (SEQ ID NO:13) and the gas vesicle protein GvpM (SEQ ID NO: 15) where the operon has a restriction site and start codon 208 on the 5′ end of the gas vesicle protein complex 210. SM, 212 is a selectable marker such as a bleomycin (Ble) resistance marker, a hygromycin resistance marker, hygromycin, the paromomycin resistance marker (aph VIIIsr) or a fluorescent fusion protein yellow fluorescent protein (YFP), a cyan fluorescent protein (CFP), a red fluorescent protein (mRFP). A stop codon and 3′ cassette restriction site 218 provides the transcription termination on the 3′UTR 214 of the single operon. Each of these components is operably linked to the next, i.e., the promoter coding sequence is operably linked to the 5′ end of the gas vesicle protein complex coding sequence encoding the gas vesicle expression proteins the gas vesicle protein coding sequencer is operably linked to the selectable marker coding sequence. The DNA construct Pro-HetGVP-SM 200 is then integrated into an expression vector, such as the pEL5 or the pSK.KmR chloroplast expression vector system and eukaryotic organisms with heterologous expression of gas vesicles are then generated including but not limited to Chaetoceros spp., Chlamydomonas reinhardii, Chlamydomonas spp., Chlorella vulgaris, Chlorella spp., Cyclotella spp., Didymosphenia spp., Dunaliella tertiolecta, Dunaliella spp., Botryococcus braunii, Botryococcus spp., Gelidium spp., Gracilaria spp., Hantscia spp., Hematococcus spp., Isochrysis spp., Laminaria spp., Navicula spp., Pleurochrysis spp. Scenedesmus spp. and Sargassum spp. Agrobacterium mediated transformation and expression (Kumar et al. Plant Science 166:731-738 (2004)) may also be used, as well as transformation using a chloroplast expression vector system or a similar system is accomplished by particle bombardment and gas vesicle protein nucleic acids are expressed resulting in gas vesicle formation and increased buoyancy where vesicles are assembled within the chloroplasts of eukaryotic algae.

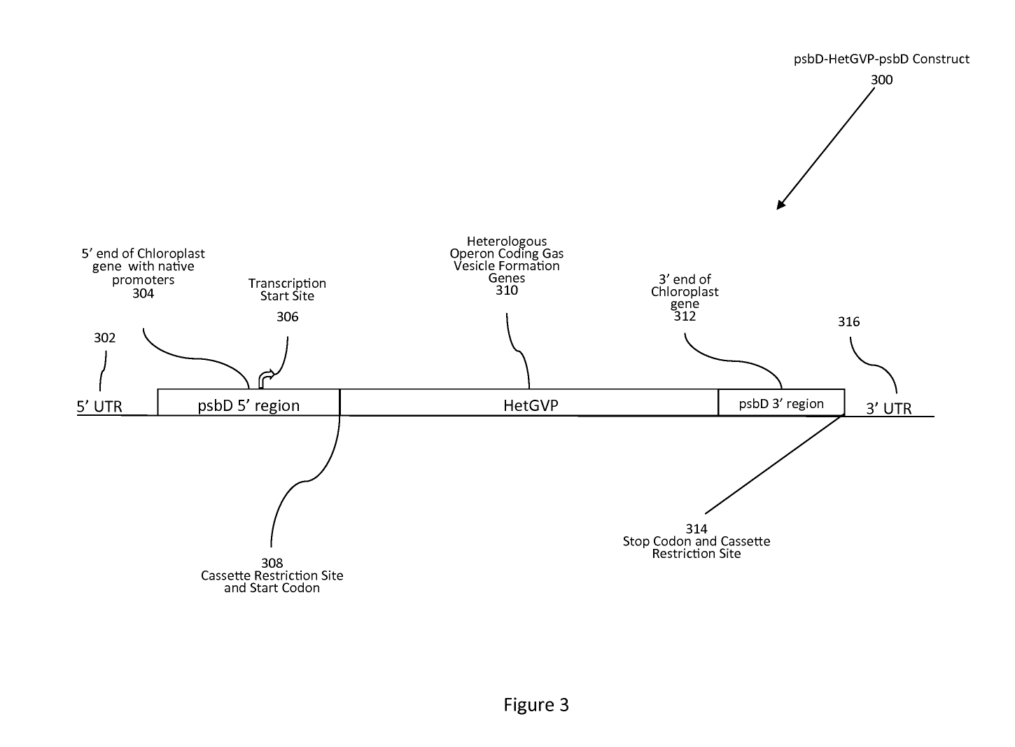

As shown in FIG. 3, a construct comprising a single operon for the homologous recombination of the transgenes into a chloroplast genome of a photosynthetic unicellular organism for the induced formation and expression of gas vesicles in a photosynthetic eukaryotic unicellular algae, such as Chlamydomonas is generally represented as psbD-HetGVP-psbD 300, where starting at the 5′ UTR 302 is the 5′ end of the psbD gene 304 which includes native promoters as well as the transcription start site 306. HetGVP 310 is a heterologous operon coding sequence comprising the synthetic gas vesicle proteins: the gas vesicle synthesis protein GvpA (SEQ ID NO. 1), the gas vesicle protein GvpO (SEQ. ID NO: 3), the gas vesicle protein GvpF (SEQ ID NO: 5), the gas vesicle protein GvpG (SEQ ID NO: 7), the gas vesicle protein GvpJ (SEQ ID NO: 9), the HAD hydrolase-like protein/gas vesicle protein GvpK (SEQ ID NO: 11), the gvpL gas vesicle protein GvpL (SEQ ID NO:13) and the gas vesicle protein GvpM (SEQ ID NO: 15) where the operon coding sequence has a restriction site and start codon 308 on the 5′ end of the operon coding sequence 310. The 3′ end of the psbD gene 312 has a stop codon and 3′ cassette restriction site 314 which provides the transcription termination on the 3′UTR 316 of the HetGVP 310 operon coding sequence. This construct allows for the integration of the heterologous operon coding genes of the heterologous operon HetGVP 310 coding genes between the 5′ and 3′ UTRs of the psbD gene and into an endogenous promoter system of the psbD gene (see Surzycki R, Cournac, Peltier G, Rochaix JD, PNAS 104(44):17548-17553 (2007)). Each of these components is operably linked to the next, i.e., the 5′ end of the psbD gene coding sequence is operably linked to the HetGVP operon coding sequence and the HetGVP operon coding sequence is operably linked to the 3′ end of the psbD gene coding sequence. The DNA construct psbD-HetGVP-psbD 300 is then integrated into an expression vector, such as the pSI105 based expression vector or pSK.KmR chloroplast expression vector system and eukaryotic organisms with heterologous expression of gas vesicles are then generated including but not limited to Chaetoceros spp., Chlamydomonas reinhardii, Chlamydomonas spp., Chlorella vulgaris, Chlorella spp., Cyclotella spp., Didymosphenia spp., Dunaliella tertiolecta, Dunaliella spp., Botryococcus braunii, Botryococcus spp., Gelidium spp., Gracilaria spp., Hantscia spp., Hematococcus spp., Isochrysis spp., Laminaria spp., Navicula spp., Pleurochrysis spp. Scenedesmus spp. and Sargassum spp.

As used herein “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

Generally, the DNA that is introduced into an organism is part of a construct. A construct is an artificially constructed segment of DNA that may be introduced into a target organism tissue or organism cell. The DNA may be a gene of interest, e.g., a coding sequence for a protein, or it may be a sequence that is capable of regulating expression of a gene, such as an antisense sequence, a sense suppression sequence, or a miRNA sequence. As used herein, “gene” refers to a segment of nucleic acid. A gene can be introduced into a genome of a species, whether from a different species or from the same species. The construct typically includes regulatory regions operably linked to the 5′ side of the DNA of interest and/or to the 3′ side of the DNA of interest. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation. A cassette containing all of these elements is also referred to herein as an expression cassette. The expression cassettes may additionally contain 5′ leader sequences in the expression cassette construct. (A leader sequence is a nucleic acid sequence containing a promoter as well as the upstream region of a gene.) The regulatory regions (i.e., promoters, transcriptional regulatory regions, translational regulatory regions, and translational termination regions) and/or the polynucleotide encoding a signal anchor may be native/analogous to the host cell or to each other. Alternatively, the regulatory regions and/or the polynucleotide encoding a signal anchor may be heterologous to the host cell or to each other. The expression cassette may additionally contain selectable marker genes. See U.S. Pat. No. 7,205,453 and U.S. Patent Application Publication Nos. 2006/0218670 and 2006/0248616. Targeting constructs are engineered DNA molecules that encode genes and flanking sequences that enable the constructs to integrate into the host genome at (targeted) locations. Publicly available restriction proteins may be used for the development of the constructs. Targeting constructs depend upon homologous recombination to find their targets.

The expression cassette or chimeric genes in the transforming vector typically have a transcriptional termination region at the opposite end from the transcription initiation regulatory region. The transcriptional termination region may normally be associated with the transcriptional initiation region from a different gene. The transcriptional termination region may be selected, particularly for stability of the mRNA, to enhance expression. Illustrative transcriptional termination regions include the NOS terminator from Agrobacterium Ti plasmid and the rice α-amylase terminator.

### Promoters

A promoter is a DNA region, which includes sequences sufficient to cause transcription of an associated (downstream) sequence. The promoter may be regulated, i.e., not constitutively acting to cause transcription of the associated sequence. If inducible, there are sequences present therein which mediate regulation of expression so that the associated sequence is transcribed only when an inducer molecule is present. The promoter may be any DNA sequence which shows transcriptional activity in the chosen cells or organisms. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley and Reynolds, Nucleic Acids Res., 15, 2343-61 (1987). Also, the location of the promoter relative to the transcription start may be optimized. Many suitable promoters for use in algae, plants, and photosynthetic bacteria are well known in the art, as are nucleotide sequences, which enhance expression of an associated expressible sequence.

While the IPTG inducible Ptrc promoter, the pEL5 translational enhancing sequence, a rbcl promoter or other chloroplast promoter, the RbcS2 promoter (SEQ ID NO: 18), the PSAD promoter (SEQ ID NO: 17) or the regulatory region upstream of the protein coding sequences are examples of promoters that may be used, a number of promoters may be used including but not limited to the RbcS2 promoter, the PSAD promoter, the NIT1 promoter, the CYC6 promoter and, prokaryotic lac and Ptrc promoters and eukaryotic based promoters. Promoters can be selected based on the desired outcome. That is, the nucleic acids can be combined with constitutive, tissue-preferred, or other promoters for expression in the host cell of interest. Translational enhancing sequences and outer membrane trafficking signal peptide sequences are assembled around NOX4 as necessary (and is species specific) for proper protein expression and localization to the outer membrane.

### Gas Vesicle Proteins

Gas vesicles are structures found in some cyanobacteria that provide buoyancy to the photosynthetic unicellular organism. The buoyancy of the unicellular organism allows the organism to stay in the upper areas of a water column to allow the organism to perform photosynthesis.

Cyanobacterial genera including but not limited to Synechococcus, Aphanizomenon, Anadaena, Gleotrichia, Oscillatoria, Halobacterium, Calothrix and Nostoc are capable of forming gas vesicles or vacuoles for buoyancy control. Any species included in the above stated genera may be genetically modified, and any other gas vesicle containing cyanobacteria, to overexpress native or heterologous gas vesicle forming proteins upon genetic induction. Overexpression in buoyant cyanobacteria may be accomplished in two different ways: the first is by cisgenic overexpression of transcription factors or regulatory proteins that function to up-regulate gas vesicle formation such as but not limited to the gas vesicle synthesis protein GvpA (SEQ ID NO. 1 or SEQ ID NO:2), the gas vesicle protein GvpO (SEQ. ID NO: 3 or SEQ ID NO:4), the gas vesicle protein GvpF (SEQ ID NO: 5 or SEQ ID NO:6), the gas vesicle protein GvpG (SEQ ID NO: 7 or SEQ ID NO:8), the gas vesicle protein GvpJ (SEQ ID NO: 9 or SEQ ID NO:10), the HAD hydrolase-like protein/gas vesicle protein GvpK (SEQ ID NO: 11 or SEQ ID NO:12), the gvpL gas vesicle protein GvpL (SEQ ID NO:13 or SEQ ID NO:14) the gas vesicle protein GvpM (SEQ ID NO: 15 or SEQ ID NO:16) and the GvpE gas vesicle protein from Haloferax volcanii. Conversely, knocking out transcriptional deactivators such as but not limited to GvpD may be used. Secondly, cisgenic or transgenic express vectors may be used to accomplish induced buoyancy by using cisgenic and or transgenic expression vectors capable of expressing endogenous or heterologous gas vesicle protein constituents in transformed cell lines, where the proteins again may include but are not limited to the gas vesicle synthesis protein GvpA (SEQ ID NO. 1 or SEQ ID NO:2), the gas vesicle protein GvpO (SEQ. ID NO: 3 or SEQ ID NO:4), the gas vesicle protein GvpF (SEQ ID NO: 5 or SEQ ID NO:6), the gas vesicle protein GvpG (SEQ ID NO: 7 or SEQ ID NO:8), the gas vesicle protein GvpJ (SEQ ID NO: 9 or SEQ ID NO:10), the HAD hydrolase-like protein/gas vesicle protein GvpK (SEQ ID NO: 11 or SEQ ID NO:12), the gvpL gas vesicle protein GvpL (SEQ ID NO:13 or SEQ ID NO:14) and the gas vesicle protein GvpM (SEQ ID NO: 15 or SEQ ID NO:16).

### Vector Construction, Transformation, and Heterologous Protein Expression

As used herein plasmid, vector or cassette refers to an extrachromosomal element often carrying genes and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with an appropriate 3′ untranslated sequence into a cell.

An example of an expression vector is the plastid or bacterial pEL5 expression vector (see Lan, EI, and Liao, JC, Metabolic Engineering 13:353-363, (2011)) or the plastid pSK.KmR expression vector (Bateman J M and Parton S, Molecular Genetics 263: 404-410 (2000)). Derivatives of the vectors described herein may be capable of stable transformation of many photosynthetic unicells, including but not limited to unicellular algae of many species, chloroplasts, photosynthetic bacteria, and single photosynthetic cells, e.g. protoplasts, derived from the green parts of plants. Vectors for stable transformation of algae, bacteria, and plants are well known in the art and can be obtained from commercial vendors. Expression vectors can be engineered to produce heterologous and/or homologous protein(s) of interest (e.g., antibodies, mating type agglutinins, etc.). Such vectors are useful for recombinantly producing the protein of interest. Such vectors are also useful to modify the natural phenotype of host cells (e.g., expressing or overexpressing a gas vesicle protein).

To construct the vector, the upstream DNA sequences of a gene expressed under control of a suitable promoter may be restriction mapped and areas important for the expression of the protein characterized. The exact location of the start codon of the gene is determined and, making use of this information and the restriction map, a vector may be designed for expression of a heterologous protein by removing the region responsible for encoding the gene's protein but leaving the upstream region found to contain the genetic material responsible for control of the gene's expression. A synthetic oligonucleotide is preferably inserted in the location where the protein sequence once was, such that any additional gene could be cloned in using restriction endonuclease sites in the synthetic oligonucleotide (i.e., a multicloning site). An unrelated gene (or coding sequence) inserted at this site would then be under the control of an extant start codon and upstream regulatory region that will drive expression of the foreign (i.e., not normally present) protein encoded by this gene. Once the gene for the foreign protein is put into a cloning vector, it can be introduced into the host organism using any of several methods, some of which might be particular to the host organism. Variations on these methods are described in the general literature. Manipulation of conditions to optimize transformation for a particular host is within the skill of the art.

The basic transformation techniques for expression in photosynthetic unicells are commonly known in the art. These methods include, for example, introduction of plasmid transformation vectors or linear DNA by use of cell injury, by use of biolistic devices, by use of a laser beam or electroporation, by microinjection, or by use of Agrobacterium tumifaciens for plasmid delivery with transgene integration or by any other method capable of introducing DNA into a host cell.

In some embodiments, biolistic plasmid transformation of the chloroplast genome can be achieved by introducing regions of chloroplast DNA flanking a desired nucleotide sequence, allowing for homologous recombination of the exogenous DNA into the target chloroplast genome. Plastid transformation is a routine and well known in the art (see U.S. Pat. Nos. 5,451,513, 5,545,817, and 5,545,818; WO 95/16783; McBride et al., Proc. Natl. Acad. Sci., USA 91:7301-7305, 1994). In some instances one to 1.5 kb flanking nucleotide sequences of chloroplast genomic DNA may be used. Using this method, point mutations in the chloroplast 16S rRNA and rps12 genes, which confer resistance to spectinomycin and streptomycin, can be utilized as selectable markers for transformation and can result in stable homoplasmic transformants, at a frequency of approximately one per 100 bombardments of target cells (Svab et al., Proc. Natl. Acad. Sci., USA 87:8526-8530, 1990).

Biolistic microprojectile-mediated transformation also can be used to introduce a polynucleotide into photosynthetic unicells for nuclear integration. This method utilizes microprojectiles such as gold or tungsten, which are coated with the desired polynucleotide by precipitation with calcium chloride, spermidine or polyethylene glycol. The microprojectile particles are accelerated at high speed into cells using a device such as the BIOLISTIC PD-1000 particle gun. Methods for the transformation using biolistic methods are well known in the art. Microprojectile mediated transformation has been used, for example, to generate a variety of transgenic organisms. Transformation of photosynthetic unicells also can be transformed using, for example, Agrobactium mediated transformation, biolistic methods as described above, protoplast transformation, electroporation of partially permeabilized cells, introduction of DNA using glass fibers, the glass bead agitation method, and the like. Transformation frequency may be increased by replacement of recessive rRNA or r-protein antibiotic resistance genes with a dominant selectable marker, including, but not limited to the bacterial aadA gene (Svab and Maiiga, Proc. Natl. Acad. Sci., USA 90:913-917, 1993).

The basic techniques used for transformation and expression in photosynthetic organisms are known in the art. These methods have been described in a number of texts for standard molecular biological manipulation (see Packer & Glaser, 3988, “Cyanobacteria”, Meth. Enzymol., Vol. 167; Weissbach & Weissbach, 1988, “Methods for plant molecular biology,” Academic Press, New York, Sambrook, Fritsch & Maniatis, 1989, “Molecular Cloning: A laboratory manual,” 2nd edition Cold Spring Harbor Laboratory Press, Cold Spring Harbor, N.Y.; and Clark M S, 1997, Plant Molecular Biology, Springer, N.Y.). These methods include, for example, biolistic devices (See for example, Sanford, Trends In Biotech. (1988) 6: 299-302, U.S. Pat. No. 4,945,050; electroporation (Fromm et al., Proc. Nat'l. Acad. Sci. (USA) (1985) 82: 5824-5828); use of a laser beam, electroporation, microinjection or any other method capable of introducing DNA into a host cell (e.g., an NVPO).

Another transformation method is described in Surzycki R, Cournac, Peltier G, Rochaix JD (2007) “Potential for hydrogen production with inducible chloroplast gene expression in Chlamydomonas.” PNAS 104(44):17548-17553. This method is replaces the chloroplast gene of the photosynthetic unicellular organism by replacing its 5′ UTR with the 5′ end of the psbD gene.

Other transformation methods are available to those skilled in the art, such as direct uptake of foreign DNA constructs (see EP 295959), techniques of electroporation (see Fromm et al. (1986) Nature (London) 319:791) or high-velocity ballistic bombardment with metal particles coated with the nucleic acid constructs (see Kline et al. (1987) Nature (London) 327:70, and see U.S. Pat. No. 4,945,050).

To confirm the presence of the transgenes in transgenic cells, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic organisms have been obtained, they may be grown to produce organisms or parts having the desired phenotype.

### Use of a Selectable Marker (SM)

A selectable marker can provide a means to obtain prokaryotic cells or plant cells or both that express the marker and, therefore, can be useful as a component of a vector. Examples of selectable markers include, but are not limited to, those that confer antimetabolite resistance, for example, dihydrofolate reductase, which confers resistance to methotrexate; neomycin phosphotransferase, which confers resistance to the aminoglycosides neomycin, kanamycin and paromycin; hygro, which confers resistance to hygromycin, trpB, which allows cells to utilize indole in place of tryptophan; hisD, which allows cells to utilize histinol in place of histidine; mannose-6-phosphate isomerase which allows cells to utilize mannose; ornithine decarboxylase, which confers resistance to the ornithine decarboxylase inhibitor, 2-(difluoromethyl)-DL-ornithine; and deaminase from Aspergillus terreus, which confers resistance to Blasticidin S. Additional selectable markers include those that confer herbicide resistance, for example, phosphinothricin acetyltransferase gene, which confers resistance to phosphinothricin, a mutant EPSPV-synthase, which confers glyphosate resistance, a mutant acetolactate synthase, which confers imidazolione or sulfonylurea resistance, a mutant psbA, which confers resistance to atrazine, or a mutant protoporphyrinogen oxidase, or other markers conferring resistance to an herbicide such as glufosinate. Selectable markers include polynucleotides that confer dihydrofolate reductase (DHFR) or neomycin resistance for eukaryotic cells and tetracycline; ampicillin resistance for prokaryotes such as E. coli; and bleomycin, gentamycin, glyphosate, hygromycin, kanamycin, methotrexate, phleomycin, phosphinotricin, spectinomycin, streptomycin, sulfonamide and sulfonylurea resistance in plants.

Fluorescent peptide (FP) fusions allow analysis of dynamic localization patterns in real time. Over the last several years, a number of different colored fluorescent peptidess have been developed and may be used in various constructs, including yellow FP (YFP), cyan FP (CFP), red FP (mRFP) and others. Some of these peptides have improved spectral properties, allowing analysis of fusion proteins for a longer period of time and permitting their use in photobleaching experiments. Others are less sensitive to pH, and other physiological parameters, making them more suitable for use in a variety of cellular contexts. Additionally, FP-tagged proteins can be used in protein-protein interaction studies by bioluminescence resonance energy transfer (BRET) or fluorescence resonance energy transfer (FRET). High-throughput analyses of FP fusion proteins in Arabidopsis have been performed by overexpressing cDNA-GFP fusions driven by strong constitutive promoters. A standard protocol is to insert the mRFP tag or marker at a default position of ten amino acids upstream of the stop codon, following methods established for Arabidopsis (Tian et al. High through put fluorescent tagging of full-length Arabidopsis gene products in plants. Plant Physiol. 135 25-38). Although useful, this approach has inherent limitations, as it does not report tissue-specificity, and overexpression of multimeric proteins may disrupt the complex. Furthermore, overexpression can lead to protein aggregation and/or mislocalization.

In order to tag a specific gene with a fluorescent peptide such as the red fluorescent protein (mRFP), usually a gene ideal for tagging has been identified through forward genetic analysis or by homology to an interesting gene from another model system. For generation of native expression constructs, full-length genomic sequence is required. For tagging of the full-length gene with an FP, the full-length gene sequence should be available, including all intron and exon sequences. A standard protocol is to insert the mRFP tag or marker at a default position of ten amino acids upstream of the stop codon, following methods known in the art established for Arabidopsis. The rationale is to avoid masking N-terminal targeting signals (such as endoplasmic reticulum (ER) retention or peroxisomal signals). In addition, by avoiding the N-terminus, disruption of N-terminal targeting sequences or transit peptides is avoided. However, choice of tag insertion is case-dependent, and it should be based on information on functional domains from database searches. If a homolog of the gene of interest has been successfully tagged in another organism, this information is also used to choose the optimal tag insertion site.

Flag tags or reporter tags/epitopes, such as artificial genes with 5′ and 3′ restriction sites and C-terminal 3X FLAG tags are another mechanism to allow for analysis of the location and presence of a gene. The C-terminal FLAG tag/epitope allows screening of transformants and analysis of protein expression by standard Western blot using commercially available anti-FLAG M2 primary antibody. 5′ ribosomal binding sites are added to each vesicle protein coding sequence or ORF such that each vesicle ORF is translated independently of the operon sequence.

### Linker

A flexible linker peptide may be placed between proteins such that the desired protein obtained. A cleavable linker peptide may also be placed between proteins such that they can be cleaved and the desired protein obtained. An example of a flexible linker may include (GSS)2.

### Transcription Terminator

The transcription termination region of the constructs is a downstream regulatory region including the stop codon TGA and the transcription terminator sequence. Alternative transcription termination regions which may be used may be native with the transcriptional initiation region, may be native with the DNA sequence of interest, or may be derived from another source. The transcription termination region may be naturally occurring, or wholly or partially synthetic. Convenient transcription termination regions are available from the Ti-plasmid of Agrobacterium tumefaciens, such as the octopine synthase and nopaline synthase transcription termination regions or from the genes for beta-phaseolin, the chemically inducible plant gene, pIN.

### Growing a Transgenic Unicellular Organism

A variety of methods are available for growing photosynthetic unicellular organisms. Cells can be successfully grown in a variety of media including agar and liquid, with shaking or mixing. Long term storage of cells can be achieved using plates and storing a 10-15° C. Cells may be stored in agar tubes, capped and grown in a cool, low light storage area. Photosynthetic unicells are usually grown in a simple medium with light as the sole energy source including in closed structures such as photobioreactors, where the environment is under strict control. A photobioreactor is a bioreactor that incorporates a light source.

While the techniques necessary for growing unicellular organisms are known in the art, an example method of growing unicells may include using a liquid culture for growth including 100 μl of 72 hr liquid culture used to inoculate 3 ml of medium in 12 well culture plates that are grown for 24 hrs in the light with shaking.

Another example may include the use of 300 ul of 72 hr liquid culture used to inoculate 5 ml of medium in 50 ml culture tubes where the unicells cultures are grown for 72 hrs under light with shaking Cultures are vortexed and photographed. Cultures are then left to settle for 10 min and photographed again.

The practice described herein employs, unless otherwise indicated, conventional techniques of chemistry, molecular biology, microbiology, recombinant DNA, genetics, immunology, cell biology, cell culture and transgenic biology, which are within the skill of the art. See e.g., Maniatis, et al., Molecular Cloning, Cold Spring Harbor Laboratory Press, Cold Spring Harbor, N.Y. (1982); Sambrook, et al., Molecular Cloning, 2nd Ed., Cold Spring Harbor Laboratory Press, Cold Spring Harbor, N.Y. (1989); Sambrook and Russell, Molecular Cloning, 3rd Ed., Cold Spring Harbor Laboratory Press, Cold Spring Harbor, N.Y. (2001); Ausubel, et al., Current Protocols in Molecular Biology, John Wiley & Sons (including periodic updates) (1992); Glover, DNA Cloning, IRL Press, Oxford (1985); Russell, Molecular biology of plants: a laboratory course manual, Cold Spring Harbor Laboratory Press, Cold Spring Harbor, N.Y. (1984); Anand, Techniques for the Analysis of Complex Genomes, Academic Press, NY (1992); Guthrie and Fink, Guide to Yeast Genetics and Molecular Biology, Academic Press, NY (1991); Harlow and Lane, Antibodies, Cold Spring Harbor Laboratory Press, Cold Spring Harbor, N.Y. (1988); Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, A. R. Liss, Inc. (1987); Immobilized Cells And Enzymes, IRL Press (1986); B. Perbal, A Practical Guide To Molecular Cloning (1984); the treatise, Methods In Enzymology, Academic Press, Inc., NY); Methods In Enzymology, Vols. 154 and 155, Wu, et al., eds.; Immunochemical Methods In Cell And Molecular Biology, Mayer and Walker, eds., Academic Press, London (1987); Handbook Of Experimental Immunology, Volumes I-IV, D. M. Weir and C. C. Blackwell, eds. (1986); Riott, Essential Immunology, 6th Edition, Blackwell Scientific Publications, Oxford (1988); Fire, et al., RNA Interference Technology: From Basic Science to Drug Development, Cambridge University Press, Cambridge (2005); Schepers, RNA Interference in Practice, Wiley VCH (2005); Engelke, RNA Interference (RNAi): The Nuts & Bolts of siRNA Technology, DNA Press (2003); Gott, RNA Interference, Editing, and Modification: Methods and Protocols (Methods in Molecular Biology), Human Press, Totowa, N.J. (2004); and Sohail, Gene Silencing by RNA Interference: Technology and Application, CRC (2004).

### EXAMPLES

The following examples are provided to illustrate further the various applications and are not intended to limit the invention beyond the limitations set forth in the appended claims.

### Example 1

### Induced Overexpression of Gas Vesicles in Cyanobacteria

In at least one embodiment is provided a cyanobacteria capable of heterologous overexpression of transcription factors or regulatory proteins that function to up-regulate gas vesicle formation such as but not limited to GvpE from Haloferax volcanii. To induce cisgenic or transgenic flotation, cyanobacteria are transformed with eight genes, the gas vesicle protein GvpA (SEQ ID NO. 1), the gas vesicle protein GvpO (SEQ. ID NO: 3), the gas vesicle protein GvpF (SEQ ID NO: 5), the gas vesicle protein GvpG (SEQ ID NO: 7), the gas vesicle protein GvpJ (SEQ ID NO: 9), the HAD hydrolase-like protein/gas vesicle protein GvpK (SEQ ID NO: 11), the gvpL gas vesicle protein GvpL (SEQ ID NO:13) and the gas vesicle protein GvpM (SEQ ID NO: 15) organized into one, two or more operons that are integrated into the host cell genome or expressed from a separate plasmid or plasmids. The gvpAOFGJKLM genes are necessary and sufficient for gas vesicle formation. gvpA (SEQ ID NO: 1) and gvpO (SEQ ID NO: 3) are expressed on a single but separate operon from gvpFGJKLM genes to assure correct expression levels. Synechococcus spp., H. salinarum, Calothrix, Anabaena flos-aquae and any other characterized gyp genes (AOFGJKLM) coding for gas vesicle protein expression and vesicle formation are used. Native homologues of these genes are overexpressed in cyanobacterial strains that possess them. Artificial gas vesicle forming genes that have been commercially synthesized and codon optimized for each species for which heterologous expression are also used.

Transgenic and cisgenic expression of the gvpAO and gvpFGJKLM genes are carried out using expression vectors based on pEL5 using the IPTG inducible Ptrc promoter and pEL5 translational enhancing sequences. Standard transformation methods such as electroporation or others are used for suitable species. gvpAO and gvpFGJKLM genes are taken from organisms such as but not limited to Synechococcus spp., H. salinarum, Calothrix spp., Anabaena flos-aquae, Aphanizomenon spp., Anadaena spp., Gleotrichia spp., Oscillatoria spp. and Nostoc spp.

Standard recombinant DNA techniques and gene synthesis methods are used to generate all constructs. The gvpAO and gvpFGJKLM CDSs for the gas vesicle protein GvpA (SEQ ID NO. 1), the gas vesicle protein GvpO (SEQ. ID NO: 3), the gas vesicle protein GvpF (SEQ ID NO: 5), the gas vesicle protein GvpG (SEQ ID NO: 7), the gas vesicle protein GvpJ (SEQ ID NO: 9), the HAD hydrolase-like protein/gas vesicle protein GvpK (SEQ ID NO: 11), the gvpL gas vesicle protein GvpL (SEQ ID NO:13) and the gas vesicle protein GvpM (SEQ ID NO: 15). The expression vector, pEL5 and its derivatives drive transcription using a truncated IPTG inducible Ptrc promoter and pEL5 translational enhancing sequences.

gvpAO and gvpFGJKLM synthetic constructs are subcloned in-frame into pEL5 with all regulatory elements as a restriction fragment. by amplification with primers that added a 5′BglII site, a 3′MscI site and removed the stop codon.

Transformation using the construct comprising the operon IPTGgvpAO and the operon gvpFGJKLM is carried out according to standard electroporation or other transformational methods.

Colonies are further screened for positive transformation via PCR targeting the transgenic operons. Genomic DNA is extracted by incubating cells at 100° C. for 5 min in 10 mM NaEDTA followed by centrifugation.

### Example 2

### Induced Overexpression of Gas Vesicles in Cyanobacteria

Example 1 is repeated for the heterologous gas vesicle expression in model and commonly used cyanobacteria that are not yet known to produce gas vesicles or vacuoles, including but not limited to Arthrospira spp. Or Spirulina spp., Synechococcus elongatus 7942, Synechococcus spp., Synechosystis spp. PCC 6803, Synechosystis spp., and Spirulina plantensis.

### Example 3

### Induced Heterologous Expression of Gas Vesicles in Eukaryotic Unicellular Algae

For the induced heterologous expression of gas vesicles in eukaryotic unicellular algae, genes from all eight gas vesicle synthesis genes (gvpAOFGJKLM) the gas vesicle protein GvpA (SEQ ID NO. 1), the gas vesicle protein GvpO (SEQ. ID NO: 3), the gas vesicle protein GvpF (SEQ ID NO: 5), the gas vesicle protein GvpG (SEQ ID NO: 7), the gas vesicle protein GvpJ (SEQ ID NO: 9), the HAD hydrolase-like protein/gas vesicle protein GvpK (SEQ ID NO: 11), the gvpL gas vesicle protein GvpL (SEQ ID NO:13) and the gas vesicle protein GvpM (SEQ ID NO: 15) are cloned from one or more of the following organisms: H. salinarum, Calothrix spp., Anabaena flos-aquae, Aphanizomenon spp., Anadaena spp., Gleotrichia spp., Oscillatoria spp. and Nostoc spp. by synthetic assembly using standard codon optimization and recombinant DNA techniques.

The genes gvpAOFGJKLM are assembled in silico into the proper operons or open reading frame (“ORF”) with promoters, ribosome binding sites and/or regulatory sequences for heterologous expression into one of the following organismic systems: Arthrospira spp./Spirulina spp., Calothrix spp., Anabaena flos-aquae, Aphanizomenon spp., Anadaena spp., Gleotrichia spp., Oscillatoria spp., Nostoc spp., Synechococcus elongates 7942, Synechococcus spp., Synechosystis spp. PCC 6803, Synechosystis spp., Spirulina plantensis, Chaetoceros spp., Chlamydomonas reinhardii, Chlamydomonas spp., Chlorella vulgaris, Chlorella spp., Cyclotella spp., Didymosphenia spp., Dunaliella tertiolecta, Dunaliella spp., Botryococcus braunii, Botryococcus spp., Gelidium spp., Gracilaria spp., Hantscia spp., Hematococcus spp., Isochrysis spp., Laminaria spp., Navicula spp., Pleurochrysis spp. and Sargassum spp. The in silico operon assembly containing all necessary vesicle proteins, selective markers, fusion tags, restriction sites, ribosome binding sites and regulatory sequences are then synthesized using a service provider such as GenScript Corporation, Piscataway, N.J. This artificial DNA construct is then subcloned or ligated into an expression vector, such as pEL5 or pSK.KmR and biolistically transformed into the chloroplast for heterologous protein expression.

Each organismic system requires 1) a nucleic acid expression vector system with species specific promoter ribosome binding sites and regulatory sequence and 2) an effective species specific transformation procedure. Many suitable promoters for use in algae are well known in the art, as are nucleotide sequences, which enhance expression of an associated expressible sequence.

Transgenic or cisgenic strains are strains are selected, screened for floatation and grown to a stationary phase on large scales where successful gas vesicle upregulation/expression is shown. Successful vesicle expression results in the floatation of cells to the culture surface where harvesting occurs via skimming. Minimal downstream processing may be necessary to sufficiently concentrate and dry the biomass. Processes occurring after induced floatation lie outside the scope of this invention.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions, and sub-combinations as are within their true spirit and scope.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate embodiment of the invention.

Moreover, though the description of the invention has included description of one or more embodiments and certain variations and modifications, other variations and modifications are within the scope of the invention (e.g., as may be within the skill and knowledge of those in the art, after understanding the present disclosure). It is intended to obtain rights which include alternative embodiments to the extent permitted, including alternate, interchangeable and/or equivalent structures, functions, ranges or acts to those claimed, whether or not such alternate, interchangeable and/or equivalent structures, functions, ranges or acts are disclosed herein, and without intending to publicly dedicate any patentable subject matter.

The use of the terms “a,” “an,” and “the,” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

We claim:

1. A DNA construct for the formation and expression or overexpression of gas vesicle protein coding sequences or vacuole protein coding sequences in photosynthetic unicells, wherein said DNA construct comprises a promoter and one operon, wherein said promoter is operably linked to said one operon, wherein said a single operon comprises gas vesicle expression protein coding sequences or vacuole expression protein coding sequences, wherein said gas vesicle expression protein coding sequences or vacuole expression protein coding sequences comprising SEQ ID NO:1, SEQ ID NO:3, SEQ ID NO:5, SEQ ID NO:7, SEQ ID NO:9, SEQ ID NO:11, SEQ ID NO:13 and SEQ ID NO:15.

2. The DNA construct of claim 1 wherein said promoter is chosen from SEQ ID NO:17 and SEQ ID NO:18.

3. The DNA construct of claim 2, wherein said DNA construct further comprises a selectable marker operably linked to the 5′ end of said promoter coding sequence.

4. The DNA construct of claim 3, wherein said DNA construct further comprises a fluorescent peptide tag operably linked to the 5′ end of said gas vesicle or vacuole expression protein coding sequence.

5. The DNA construct of claim 2, wherein said DNA construct further comprises a fluorescent peptide tag operably linked to the 3′ end of said gas vesicle or vacuole expression protein coding sequence.

6. A transgenic photosynthetic unicellular organism having said DNA construct of claim 1 stably integrated into a photosynthetic unicellular organism's nuclear genome or said organism's chloroplast genome under conditions suitable for an expression of said DNA construct in said photosynthetic unicellular organism, wherein the DNA construct expresses vesicle or vacuole proteins in said photosynthetic unicellular organism.

7. A transgenic photosynthetic unicellular organism having said DNA construct of claim 2 stably integrated into a photosynthetic unicellular organism's nuclear genome or said organism's chloroplast genome under conditions suitable for an expression of said DNA construct in said photosynthetic unicellular organism, wherein the DNA construct expresses vesicle or vacuole proteins in said photosynthetic unicellular organism.

8. A transgenic photosynthetic unicellular organism having said DNA construct of claim 3 stably integrated into a photosynthetic unicellular organism's nuclear genome or said organism's chloroplast genome under conditions suitable for an expression of said DNA construct in said photosynthetic unicellular organism, wherein the DNA construct expresses vesicle or vacuole proteins in said photosynthetic unicellular organism.

9. A transgenic photosynthetic unicellular organism having said DNA construct of claim 4 stably integrated into a photosynthetic unicellular organism's nuclear genome or said organism's chloroplast genome under conditions suitable for an expression of said DNA construct in said photosynthetic unicellular organism, wherein the DNA construct expresses vesicle or vacuole proteins in said photosynthetic unicellular organism.

10. A transgenic photosynthetic unicellular organism having said DNA construct of claim 5 stably integrated into a photosynthetic unicellular organism's nuclear genome or said organism's chloroplast genome under conditions suitable for an expression of said DNA construct in said photosynthetic unicellular organism, wherein the DNA construct expresses vesicle or vacuole proteins in said photosynthetic unicellular organism.

11. A method for producing gas vesicle or vacuole formation and expression or overexpression proteins in a photosynthetic unicellular organism which comprises growing a photosynthetic unicellular organism having said DNA construct of claim 1 stably integrated into said organism's nuclear genome or said organism's chloroplast genome under conditions suitable for an expression of the DNA construct in a photosynthetic unicellular organism, wherein the DNA construct expresses a gas vesicle or gas vacuole protein in said photosynthetic unicellular organism.

12. A method for producing gas vesicle or vacuole formation and expression or overexpression proteins in a photosynthetic unicellular organism which comprises growing a photosynthetic unicellular organism having said DNA construct of claim 2 stably integrated into said organism's nuclear genome or said organism's chloroplast genome under conditions suitable for an expression of the DNA construct in a photosynthetic unicellular organism, wherein the DNA construct expresses a gas vesicle or gas vacuole protein in said photosynthetic unicellular organism.

13. A method for producing gas vesicle or vacuole formation and expression or overexpression proteins in a photosynthetic unicellular organism which comprises growing a photosynthetic unicellular organism having said DNA construct of claim 3 stably integrated into said organism's nuclear genome or said organism's chloroplast genome under conditions suitable for an expression of the DNA construct in a photosynthetic unicellular organism, wherein the DNA construct expresses a gas vesicle or gas vacuole protein in said photosynthetic unicellular organism.

14. A method for producing gas vesicle or vacuole formation and expression or overexpression proteins in a photosynthetic unicellular organism which comprises growing a photosynthetic unicellular organism having said DNA construct of claim 4 stably integrated into said organism's nuclear genome or said organism's chloroplast genome under conditions suitable for an expression of the DNA construct in a photosynthetic unicellular organism, wherein the DNA construct expresses a gas vesicle or gas vacuole protein in said photosynthetic unicellular organism.

15. A method for producing gas vesicle or vacuole formation and expression or overexpression proteins in a photosynthetic unicellular organism which comprises growing a photosynthetic unicellular organism having said DNA construct of claim 5 stably integrated into said organism's nuclear genome or said organism's chloroplast genome under conditions suitable for an expression of the DNA construct in a photosynthetic unicellular organism, wherein the DNA construct expresses a gas vesicle or gas vacuole protein in said photosynthetic unicellular organism.

16. A DNA construct for the formation and expression or overexpression of gas vesicle protein coding sequences or vacuole protein coding sequences in photosynthetic unicells, wherein said DNA construct comprises a first promoter and first operon, and a second promoter and a second operon wherein said first promoter is operably linked to said first operon, and said second promoter is operably linked to said second operon wherein said first operon comprises gas vesicle expression protein coding sequences or vacuole expression protein coding sequences comprising SEQ ID NO:1, SEQ ID NO:3, and wherein said second operon comprises gas vesicle expression protein coding sequences or vacuole expression protein coding sequences comprising SEQ ID NO:5, SEQ ID NO:7, SEQ ID NO:9, SEQ ID NO:11, SEQ ID NO:13 and SEQ ID NO:15.

17. The DNA construct of claim 16 wherein said promoter is chosen from SEQ ID NO:17 and SEQ ID NO:18.

18. The DNA construct of claim 16, wherein said DNA construct further comprises a first selectable marker operably linked to the 3′ end of said first operon coding sequence and a second selectable marker operably linked to the 3′ end of said second operon coding sequence.

19. The DNA construct of claim 18, wherein said DNA construct further comprises a fluorescent peptide tag operably linked to the 3′ end of said first operon protein coding sequence and a second fluorescent peptide tag operably linked to the 3′ end of said second operon protein coding sequence.

20. A transgenic photosynthetic unicellular organism having said DNA construct of claim 16 stably integrated into a photosynthetic unicellular organism's nuclear genome or said organism's chloroplast genome under conditions suitable for an expression of said DNA construct in said photosynthetic unicellular organism, wherein the DNA construct expresses vesicle or vacuole proteins in said photosynthetic unicellular organism.

21. A transgenic photosynthetic unicellular organism having said DNA construct of claim 17 stably integrated into a photosynthetic unicellular organism's nuclear genome or said organism's chloroplast genome under conditions suitable for an expression of said DNA construct in said photosynthetic unicellular organism, wherein the DNA construct expresses vesicle or vacuole proteins in said photosynthetic unicellular organism.

22. A transgenic photosynthetic unicellular organism having said DNA construct of claim 18 stably integrated into a photosynthetic unicellular organism's nuclear genome or said organism's chloroplast genome under conditions suitable for an expression of said DNA construct in said photosynthetic unicellular organism, wherein the DNA construct expresses vesicle or vacuole proteins in said photosynthetic unicellular organism.

23. A transgenic photosynthetic unicellular organism having said DNA construct of claim 19 stably integrated into a photosynthetic unicellular organism's nuclear genome or said organism's chloroplast genome under conditions suitable for an expression of said DNA construct in said photosynthetic unicellular organism, wherein the DNA construct expresses vesicle or vacuole proteins in said photosynthetic unicellular organism.

24. A method for producing gas vesicle or vacuole formation and expression or overexpression proteins in a photosynthetic unicellular organism which comprises growing a photosynthetic unicellular organism having said DNA construct of claim 16 stably integrated into said organism's nuclear genome or said organism's chloroplast genome under conditions suitable for an expression of the DNA construct in a photosynthetic unicellular organism, wherein the DNA construct expresses a gas vesicle or gas vacuole protein in said photosynthetic unicellular organism.

25. A method for producing gas vesicle or vacuole formation and expression or overexpression proteins in a photosynthetic unicellular organism which comprises growing a photosynthetic unicellular organism having said DNA construct of claim 17 stably integrated into said organism's nuclear genome or said organism's chloroplast genome under conditions suitable for an expression of the DNA construct in a photosynthetic unicellular organism, wherein the DNA construct expresses a gas vesicle or gas vacuole protein in said photosynthetic unicellular organism.

26. A method for producing gas vesicle or vacuole formation and expression or overexpression proteins in a photosynthetic unicellular organism which comprises growing a photosynthetic unicellular organism having said DNA construct of claim 18 stably integrated into said organism's nuclear genome or said organism's chloroplast genome under conditions suitable for an expression of the DNA construct in a photosynthetic unicellular organism, wherein the DNA construct expresses a gas vesicle or gas vacuole protein in said photosynthetic unicellular organism.

27. A method for producing gas vesicle or vacuole formation and expression or overexpression proteins in a photosynthetic unicellular organism which comprises growing a photosynthetic unicellular organism having said DNA construct of claim 19 stably integrated into said organism's nuclear genome or said organism's chloroplast genome under conditions suitable for an expression of the DNA construct in a photosynthetic unicellular organism, wherein the DNA construct expresses a gas vesicle or gas vacuole protein in said photosynthetic unicellular organism.

28. A DNA construct for the formation and expression or overexpression of gas vesicle protein coding sequences or vacuole protein coding sequences in photosynthetic unicellular organisms, wherein said DNA construct comprises the 5′ and 3′ UTRs of a gene of a chloroplast genome operably linked to the 5′ and 3′ end of heterologous operon coding sequence, wherein said gene of a chloroplast genome is a psbD gene and wherein said heterologous operon coding sequences comprises SEQ ID NO:1, SEQ ID NO:3, SEQ ID NO:5, SEQ ID NO:7, SEQ ID NO:9, SEQ ID NO:11, SEQ ID NO:13 and SEQ ID NO:15.

29. A transgenic photosynthetic unicellular organism having said DNA construct of claim 28 stably integrated into a photosynthetic unicellular organism's nuclear genome or said organism's chloroplast genome under conditions suitable for an expression of said DNA construct in said photosynthetic unicellular organism, wherein the DNA construct expresses vesicle or vacuole proteins in said photosynthetic unicellular organism.

30. A method for producing gas vesicle or vacuole formation and expression or overexpression proteins in a photosynthetic unicellular organism which comprises growing a photosynthetic unicellular organism having said DNA construct of claim 28 stably integrated into said organism's nuclear genome or said organism's chloroplast genome under conditions suitable for an expression of the DNA construct in a photosynthetic unicellular organism, wherein the DNA construct expresses a gas vesicle or gas vacuole protein in said photosynthetic unicellular organism.
